(19) United States
(12) Patent Application Publication
(10) Pub. No.: US 2022/0273519 A1
(43) Pub. Date: Sep. 1, 2022

(54) KITS AND METHODS FOR PREPARING PLASMA INJECTATE BIOSTIMULATOR

(71) Applicant: Riam Shammaa, Toronto (CA)

(72) Inventor: Riam Shammaa, Toronto (CA)

(21) Appl. No.: 17/673,514

(22) Filed: Feb. 16, 2022

Related U.S. Application Data
(60) Provisional application No. 63/155,085, filed on Mar. 1, 2021.

(51) Int. Cl. A61J 1/20; A61B 5/15; A61K 35/19; A61K 35/16; B01L 3/00; B01D 21/26

(52) U.S. Cl. CPC A61J 1/2096; A61B 5/150755; A61B 5/15003; A61B 5/150305; A61K 35/19; A61K 35/16; B01L 3/5021; B01D 21/262

(57) ABSTRACT

Kits and methods for preparing a plasma injectate is provided. The plasma injectate is used as a biostimulator.

### RELATED APPLICATIONS

This application claims the benefit of U.S. provisional application No. 63/155,085 filed on Mar. 1, 2021, the entire content of which is incorporated herein by reference.

### FIELD

The present disclosure generally relates to the field of platelet-rich plasma therapy, in particular kits for preparing a plasma injectate as well of methods of preparing plasma injectate for use as a biostimulator.

### BACKGROUND

Platelet-rich plasma therapy is a form of regenerative medicine that harnesses the body's abilities to heal itself and amplifies the natural growth factors the body uses to heal tissue. Plasma is the liquid portion of whole blood. It is composed largely of water and proteins, and it provides a medium for various cells including platelets to circulate through the body. Platelets, also called thrombocytes, are blood cells that cause blood clots and other necessary growth healing functions. The platelet activation pathway plays a key role in the body's natural healing process.

Platelet-rich plasma (PRP) therapy uses injections of a concentration of a patient's own platelets to accelerate the healing of injured tendons, ligaments, muscles and joints. PRP injections require collection of a patient's own blood. As well, PRP injections require activated platelets. Improved methods and tools for PRP injections are needed to standardize the therapy as well as to improve efficiency.

### SUMMARY

In one aspect, there is provided a kit for preparing a plasma injectate for, the kit comprising: a) at least one blood collection container, each containing an anticoagulant, for receiving a blood sample; b) a calcium chloride solution, for preparing an activator solution; c) an activator container for loading the activator solution, the activator container having a first volume; and d) an injectate syringe having a second volume that is larger than the first volume, the injectate syringe for fluid connection with the activator container to transfer at least a portion of the activator solution to the injectate syringe.

In another aspect, there is provided a method for preparing a plasma injectate using the kit described herein, the plasma injectate for administration to a patient, the method comprising: loading the activator container with the activator solution; collecting whole blood samples from the patient in the two blood collection containers; centrifuging the whole blood samples; extracting the platelet plasma layer from the centrifuged blood samples into the injectate syringe; connecting the injectate syringe having the platelet plasma to the loaded activator syringe; and transferring at least a portion of the activator solution to the injectate syringe.

In this respect, before explaining at least one embodiment in detail, it is to be understood that the embodiments are not limited in application to the details of construction and to the arrangements of the components set forth in the following description or illustrated in the drawings. Also, it is to be understood that the phraseology and terminology employed herein are for the purpose of description and should not be regarded as limiting.

Many further features and combinations thereof concerning embodiments described herein will appear to those skilled in the art following a reading of the instant disclosure.

### DESCRIPTION OF THE FIGURES

Embodiments of devices, apparatus, methods, and kits are described throughout reference to the drawings.

FIG. 1 shows an exemplary kit for preparing plasma injectate.

FIG. 2 shows the preparation of an activator solution.

FIG. 3A shows collecting a blood sample from a patient. FIG. 3B shows centrifuging blood samples.

FIG. 4A shows preparing centrifuged blood sample for plasma collection. FIG. 4B shows collecting plasma from centrifuged blood sample.

FIG. 5 shows a syringe configuration for activation of a plasma injectate.

FIG. 6 shows an embodiment of a tube having upper and lower chambers separated by a barrier.

### DETAILED DESCRIPTION

Numerous details are set forth to provide an understanding of the examples described herein. The examples may be practiced without these details. The description is not to be considered as limited to the scope of the examples described herein.

The present disclosure provides for kits and methods for the efficient preparation of an activated plasma injectate. As used herein, “plasma injectate” refers to a biostimulator injectate for administration to a patent. As used herein, a “biostimulator” refers to non-toxic, natural substances useful in stimulation of healing and cellular synthesis in animals. A plasma injectate comprises concentrated and activated platelets obtained from the same patient. In one embodiment, the plasma injectate comprises autologous platelets. A plasma injectate can have anti-inflammatory or pro-inflammatory properties depending on how the injectate is prepared. A plasma injectate having anti-inflammatory properties is useful in treating a patient, for example, in collagen regeneration. A plasma injectate having pro-inflammatory properties is useful in enhancing healing in a patient.

In some embodiments, the plasma injectate prepared using the kits and methods described herein is used in improving skin quality or skin healing. In some embodiments, the plasma injectate prepared using the kits and methods described herein is used in joint or musculoskeletal repair. In some embodiments, the plasma injectate prepared using the kits and methods described herein is used in wound healing and tissue repair.

In some embodiments, the plasma injectate prepared using the kits and methods described herein is used in biostimulator therapy in the context of dentistry, cardiac surgery, ophthalmology, oral and maxillofacial surgery, orthopedic surgery, plastic surgery, sports medicine and/or cosmetic medicine.

The kits and methods described herein allow for efficient preparation of plasma injectate. The components of the kits are selected for the efficient and accurate preparation of activated plasma injectate with minimal waste, thereby reducing costs and at the same time reducing the risk of human error or contamination. The kits and methods described herein also allow for consistent and standardized preparation of activated plasma injectate preparation.

Kits for Preparing Plasma Injectate

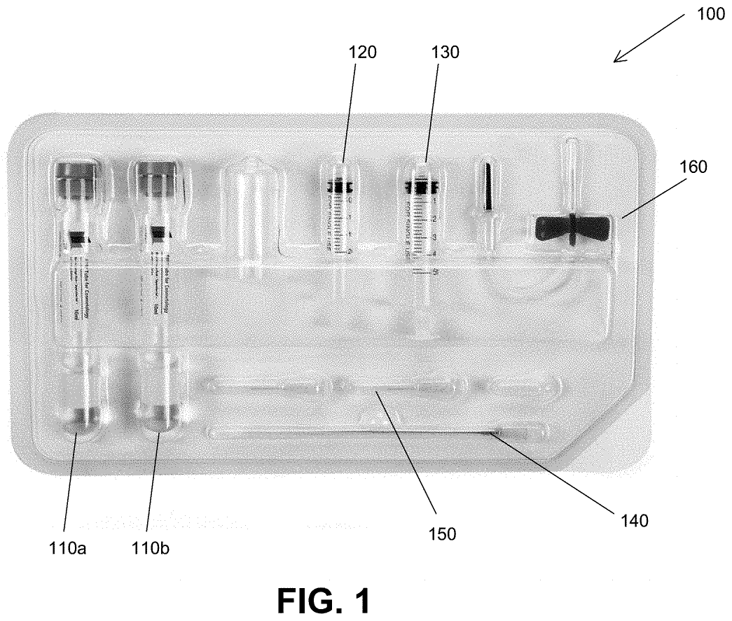

Turning to FIG. 1, an exemplary kit 100 for preparing a plasma injectate is shown. In some embodiments, the kit has at least one blood collection container. In preferred embodiments, the kit has two blood collection containers 110a, 110b, each containing a premeasured amount of anticoagulant. Whole blood samples from the patient is collected in the blood collection containers. In some embodiment, the anticoagulant is ACD-A, Sodium Citrate or heparin. Other anticoagulants can also be used so long as it does not prevent the subsequent activation of the platelets. In some embodiments, the blood collection container further contains a premeasured amount of calcium chloride solution.

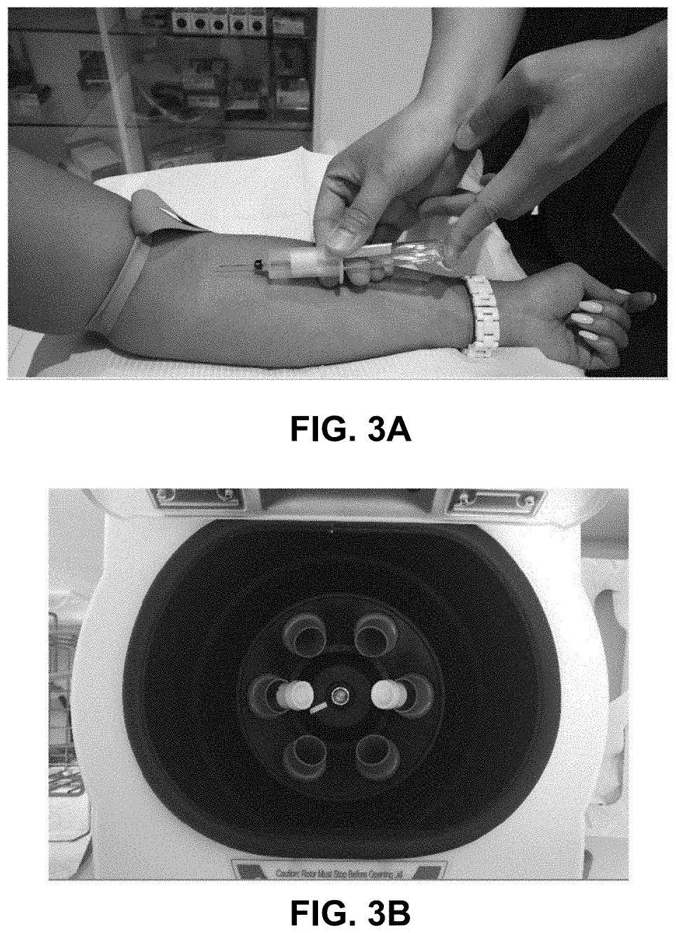

In some embodiments, the blood collection container containing the collected whole blood sample is centrifuged directly. In some embodiments, the kit further comprises one or more plasma tubes for centrifuging the collected whole blood sample. In preferred embodiments, an even number of blood collection containers and/or plasma tubes are provided to allow for counterbalanced placement of the blood collection containers and/or plasma tubes in the centrifuge (see FIG. 3B).

The kit includes a premeasured amount of calcium chloride solution for preparing an activator solution. In some embodiments, the kit includes a premade activator solution. As used herein “activator solution” refers to a solution containing a concentration of calcium ions for triggering the platelet activation pathway of platelets in a sample or in an injectate. In some embodiments, the calcium chloride solution or the activator solution comprises 5-12% calcium chloride or 50 mg-120 mg/ml calcium chloride, In some embodiments, the calcium chloride solution or the activator solution comprises 5% calcium chloride (50 mg/ml), 6% calcium chloride (60 mg/ml), 7% calcium chloride (70 mg/ml), 8% calcium chloride (80 mg/ml), 9% calcium chloride (90 mg/ml), 10% calcium chloride (100 mg/ml), 11% calcium chloride (110 mg/ml), or 12% calcium chloride (120 mg/ml). In one embodiment, the calcium chloride solution or the activator solution comprises 10% calcium chloride (100 mg/ml). In some embodiments, the activator solution comprises calcium chloride solution and a biocompatible solution or buffer. In one embodiment, the biocompatible solution or buffer is saline. In some embodiments, the kit has a calcium chloride solution having a concentration of 50 to 250 mg/ml. In one embodiment, the calcium chloride solution has a concentration of 100 mg/ml. In some embodiments, 0.5-2 ml of the calcium chloride solution is included in the kit. In one embodiment, 1 ml of the calcium chloride solution is included in the kit. In some embodiments, the kit further includes a bag of saline to dilute the calcium chloride solution into an activator solution. The premeasured volumes and concentrations of the calcium chloride solution allow for efficient preparation of an activator solution having ideal concentration of calcium ions for activating platelets.

The kit contains an activator container 120 for preparing the activator solution, and an injectate syringe 130 for preparing the plasma injectate. In some embodiments, the activator container is a syringe. The activator container is coupled to the injectate syringe to allow for transfer of fluid between the two. In some embodiments, the kit contains an adaptor 160 to couple the injectate syringe to the activator container. In one embodiment, the adaptor is a Luer lock adapter.

In use, after the activator container is loaded with the activator solution and the injectate syringe is loaded with plasma from the patient, the activator container and the injectate syringe are coupled together, and the activator solution is transferred from the activator container to the injectate syringe. Ideally the injectate syringe 130 has a larger volume than the activator container 120. The amount of activator solution transferred to the injectate syringe is dependent on the amount of plasma loaded in the injectate syringe. The greater the amount of plasma present, proportionally more activator solution is transferred.

In some embodiments, the kit includes at least one extraction needle 140. The extraction needle is coupled to the injectate syringe to withdraw plasma (containing platelets) from the centrifuged blood sample. Where the whole blood samples are centrifuged in an air-tight container, a vent is needed to withdraw the plasma from the sample container. Accordingly, in some embodiments, the kit includes one or more venting needles 150 to create a vent in the air-tight sample container.

In some embodiments, the kit further includes at least one venous needle for collecting whole blood from the patient and for coupling to the blood collection containers 110a, 110b.

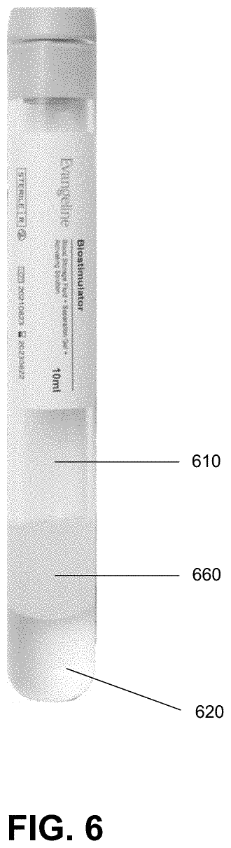

In an alternate embodiment shown in FIG. 6, the blood collection container and the activator container comprise different portions of the same tube. A centrifuge tube is separated by a barrier 660 into an upper chamber 610 and a lower chamber 620. The upper chamber 610 comprise the blood collection container for receiving the blood sample and contains the anticoagulant. The lower chamber 620 comprise the activator container for receiving the activator solution. In one embodiment, the lower chamber is pre-loaded with the activator solution. In an alternate embodiment, the upper chamber is pre-loaded with the activator solution, and the lower chamber is either left empty or filled with a separating gel.

In some embodiments, the barrier is semi-permeable. In one embodiment, the barrier breaks when the tube is centrifuged, allowing the contents of the upper and lower chamber to mix. In one embodiment, the barrier contains gaps which allow the contents of the upper or lower chamber to flow through when centrifuged. In one embodiment, the barrier is a separating gel with a weakly sealed port at one side, which releases when centrifuged allowing the contents of the upper or lower chamber to flow through.

In one preferred embodiment, the kits comprises:

i. the two blood collection tubes, each containing 1 cc per tube ACD-A;

ii. 1 ml of Calcium Chloride at a concentration of 100 mg/ml;

iii. 3 ml activator syringe;

iv. 10 ml injectate syringe;

v. two 3.5 inch 18 gauge needles;

vi. a Luer lock adapter;

vii. the two venous needles; and

viii. two 1.5 inch blunt fill needles.

In another preferred embodiment, the the kits comprises:

i. the two blood collection tubes, each containing 1 cc per tube ACD-A;

ii. 1 ml of Calcium Chloride at a concentration of 100 mg/ml;

iii. 2 ml activator syringe;

iv. 5 ml injectate syringe;

v. a 3.5 inch 18 gauge needle;

vi. the adapter; and

vii. the two venous needles.

In some embodiments, components of the kit are packaged together in a blister pack to avoid contamination during storage. In one embodiment, blood collection containers, activator container, injectate syringe, needles, and adapters are packed together in a blister pack.

Methods for Preparing Plasma Injectate

The present disclosure provides for methods for the efficient preparation of an activated plasma injectate using the kits described herein.

In some embodiments, the activator solution is prepared by diluting the calcium chloride solution with saline. In some embodiments, the calcium chloride solution is the activator solution. In one embodiment, the calcium chloride solution or the activator solution comprises 10% calcium chloride or 100 mg/ml calcium chloride. The activator solution is then loaded into the activator container. In some embodiments, the activator solution is prepared prior to collecting whole blood samples from a patient. In some embodiments, the activator solution is prepared at the same time as collecting whole blood samples from a patient and centrifuging same. In some embodiments, the activator solution is prepared after collecting whole blood samples from a patient.

Whole blood sample is collected from a patient. In some embodiments, whole blood sample is collected from a vein of a patient. The collected whole blood samples are centrifuged to isolate the plasma layer, containing the patient's platelets, from the rest of the sample. In some embodiments, the whole blood sample is centrifuged for about 7 min to prepare a plasma injectate having pro-inflammatory activity. In other embodiments, the whole blood sample is centrifuged for about 14 min to prepare a plasma injectate having anti-inflammatory activity. In yet other embodiments, the whole blood sample is first centrifuged for about 7 min and a portion is removed to prepare a first plasma injectate having pro-inflammatory activity, and the remaining sample is further centrifuged for about another 7 min to prepare a second plasma injectate having anti-inflammatory activity.

In some embodiments, the plasma is collected by withdrawing or extracting the plasma layer from the centrifuged blood sample using an injectate syringe and needle. The injectate syringe containing the plasma and the activator container containing the activator solution are connected together (for example, by using an adapting) to transfer a predetermined amount of the activator solution to the injectate syringe. After sufficient mixing, the calcium ions in the activator solution activates the platelets in the plasma, producing an activated plasma injectate.

The activated plasma is then injected using the injectate to a target location in the patient. Example target locations include, but are not limited to: an area of skin, injured tissue, muscle, joints, connective tissue, tendons and/or ligaments, or organs.

### EXAMPLES

The following examples illustrate certain embodiments addressing specific design requirements and are not intended to limit the embodiments described elsewhere in this disclosure.

### Example 1—Kit for Anti-Inflammatory (Collagen Regeneration) and Pro-Inflammatory (Healing) Yield

Components:
plasma tubes
1 cc per tube ACDA/or Sodium Citrate or heparin, or any type of anticoagulant
1 ml of Calcium Chloride 100 mg/ml vial (preserved in Benzyl alcohol)
two vacutainers
two venous needles
two 3.5 inch 18 gauge needles
two 1.5 inch blunt fill needles
gauze
alcohol swabs
saline bag (optional)
adapter
Protocol explaining exactly how to use the kit

Advantages. The combination allows for the optimal most efficient preparation of Platelet Rich Matrix (PRM) from Platelet Rich Plasma (PRP) in a very short time. The calcium chloride activates the platelets in the plasma releasing all the healing factors including CTGF, VEGEF, TGF Beta, EGF, FGF . . . etc. The kit allows for different concentrations of calcium chloride to be adjusted according to the volume to have the most optimal calcium chloride concentration for the optimal activation of the platelets, this better outcome whether for collagen regeneration/boosting (anti-inflammatory) or healing (pro-inflammatory)

### Example 2—PRProtocol Outline

A protocol was developed for efficient preparation of a plasma injectate comprising Platelet Rich Matrix (PRM) from Platelet Rich Plasma (PRP), referred to herein as “PRProtocol”.

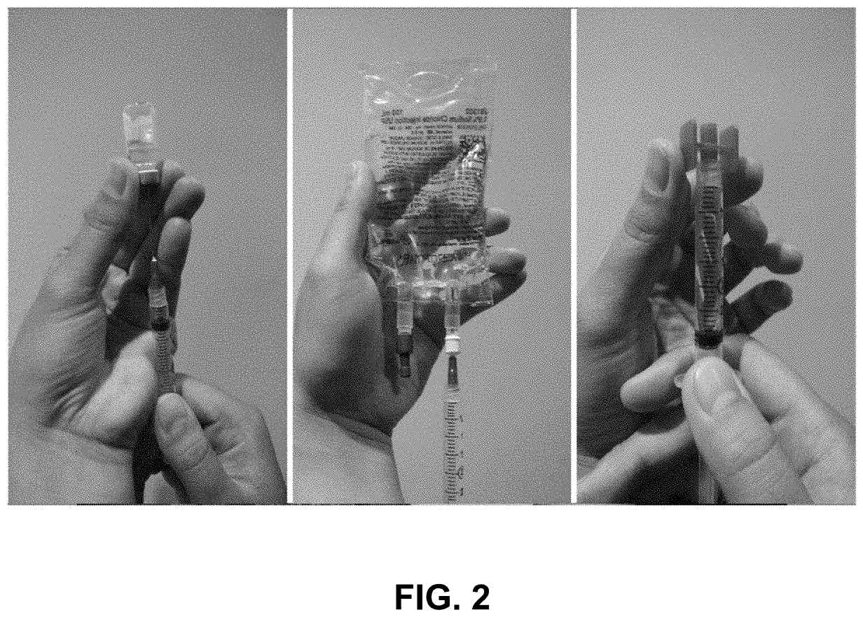

Step 1. Prepare Activator Solution (10% Calcium Chloride), see FIG. 2. Draw 0.2 ml of Calcium Chloride (CaCl) into a 3 ml syringe. Then draw 1.8 ml of sterile Normal Saline (NS) in with CaCl in the 3 ml syringe. Connect x1 RED female-to-female Luer lock adapter to the 3 ml and set aside.

Step 2. Draw venous blood into 2 ACD-A collection tubes (8.5 ml each), see FIG. 3A.

Step 3. Place the collection tubes opposite of each other in the centrifuged for balanced weight, see FIG. 3B. Centrifuge for a) 7 minutes for pro-inflammatory PRP; or b) 14 minutes for anti-inflammatory PRP.

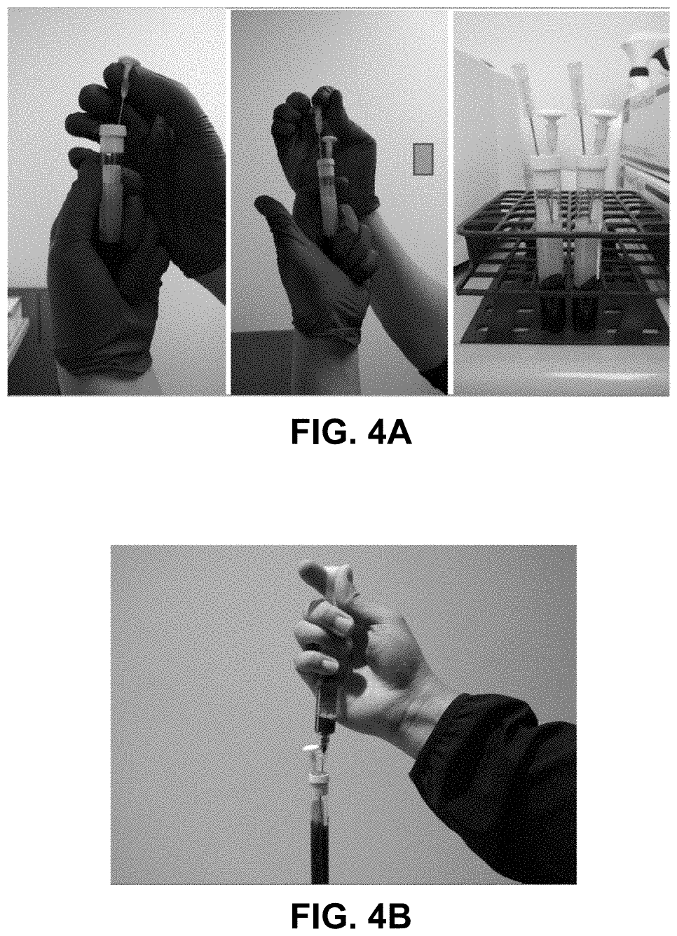

Step 4. Remove tubes with care and place upright in tube rack. Insert x1 venting needle in each collection tube. Insert one 18 G spinal needle in each collection tube, ensuring that the needle tip is roughly 5 cm above the buffy coat line. See FIG. 4A

Step 5. Connect a 10 ml syringe to the 18 G spinal needle and slowly draw the PRP from the collection tube, re-connect and repeat with 2nd tube.

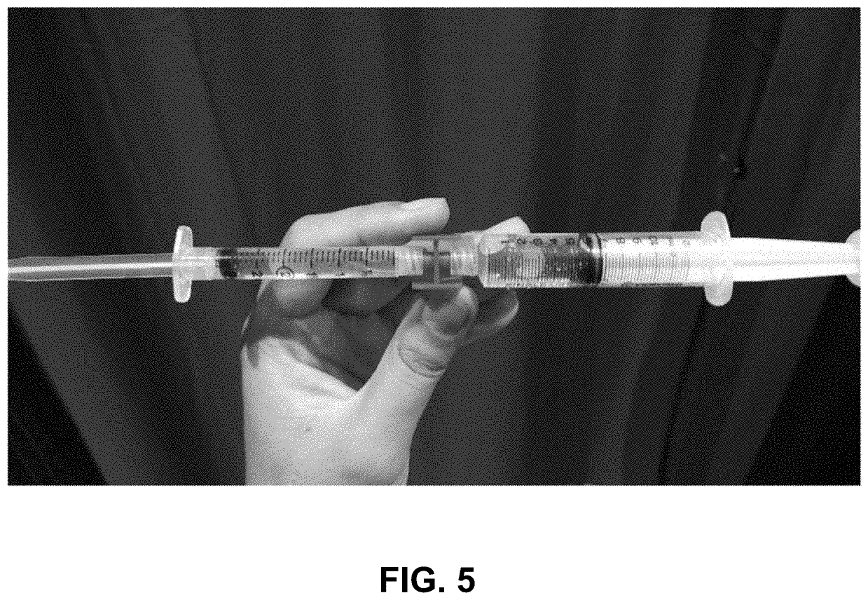

Step 6. Connect the 3 ml syringe containing the activator solution (CaCl+NS) and the 10 ml plasma injectate syringe via RED adapter, see FIG. 5. Transfer at least a portion of the activator solution to the plasma injectate syringe. The following scale is used for determining the amount of activator solution needed for transfer:

a. 0.5 ml CaCl-4 ml plasma

b. 0.6 ml CaCl-5 ml plasma

c. 0.7 ml CaCl-6 ml plasma

d. 0.8 ml CaCl-7 ml plasma

e. 0.9 ml CaCl-8 ml plasma

f. 1 ml CaCl-9 ml plasma

Step 7. Remove the 3 ml syringe. Invert the activated injectate 7-8 times, then inject the activated injectate at a desired target location in a patient.

### Example 3—Detailed Methods, Data and Discussion of PRProtocol

The objective of this study was to evaluate parameters associated with the platelet concentrates (PRP) produced by PRProtocol process. It was hypothesized that the PRProtocol will yield anti-inflammatory properties (leukocyte poor, HCT poor) that can cause collagen proliferation in the dermis leading to improved skin quality, as well as pro-inflammatory properties (Leukocyte rich, HCT poor) that can cause healing leading to leukocyte dependent repair and the production of IL-10, IL6 and VEGF according to PAW classification.

### Methods

All studies were conducted within Quality labs at University of Montreal. Up to 40 ml of human whole blood was obtained from each of 5 donors following informed consent. Donors were referenced only by assigned code numbers. Blood was drawn into a 40 cc syringe that had been preloaded with anticoagulant. An ETDA tube was drawn for baseline comparison.

Each PRProtocol was conducted from 20 ml of ACD-A anti-coagulated blood samples. Following the first centrifugation (7 minutes), the platelet plasma layer was withdrawn until the pallet approximately 5 ml of PRP recovered. The recovered platelet plasma was transferred to a tube along with 0.2 ml of ACD-A. For the second centrifugation (14 minutes total), approximately 7 ml of PRP recovered. The recovered platelet plasma was transferred to a tube along with 0.2 ml of ACD-A.

Platelet Concentration Factor. Complete blood counts (CBCs) were performed using a 3-part differential hematology analyzer to quantify the platelets contained within the start sample and platelet concentrates. The platelet concentration factor, which is the ratio of the concentration of platelets in the platelet concentrate product to the concentration of platelets in the start sample (adjusted for dilution with anticoagulant), was determined. CBC was tested according to BSR TM-076 Coulter Ac-T diff 2 Hematology Analyzer.

The platelet concentration factor (PCF) was derived as the ratio of the platelet count in the platelet concentrate (PC) to the platelet count in baseline sample (adjusted for dilution with anticoagulant) (BL): PCF=PC/BL.

Results are summarized in Table 1.7 showing observations by donor, mean platelet concentration factor and standard deviation.

Platelet Yield. CBC was performed using a hematology analyzer to quantify the platelets contained within start sample and platelet concentrates. The platelet yield, which is the ratio of the number of platelets in the platelet concentrate product to the number of platelets in the start sample, was determined.

The platelet yield (PY) was derived as the ratio of the platelet count in the platelet concentrate (PC) times the volume of the platelet concentrate (VPC) to the platelet count in the baseline sample (adjusted for dilution with anticoagulant) (BL) times the volume of the sample processed (VBL): PY=(PC*VPC)/(BL*VBL).

A two tailed, paired t-Test was used to compare the mean PLT yield for 7 minutes PRP and 14 minutes PRP.

pH of Platelet Concentrate. Sample pH was measured in platelet concentrates. The testing was conducted on a blood gas analyzer according to Standard Operating Procedure: TM-018 Blood pH. Product pH observations, per donor, from one sample of each donor PRP are shown in Table 1.8 along with means and standard deviations.

Leukocyte, Erythrocyte and Platelet Counts. CBC was performed using a hematology analyzer for start sample and platelet concentrates. The Leukocyte, Platelet counts, Erythrocyte (RBC), and calculated hematocrit (hct) were recorded for each sample. CBC was tested according to BSR TM076 Coulter Ac-T diff 2 Hematology Analyzer.

Results are summarized in Tables 1.4-1.6 showing data by donor, with calculated mean and standard deviation. A two tailed, paired t-Test was used to compare the 7 minutes PRP and 15 minutes PRP mean yields for Mononuclear Cells, Granulocytes, and RBC.

Data

Data from the PRProtocol are provided in Tables 1.1-1.8 below.

TABLE 1.1
Hematology data: Baseline

| Sample | WBC × | MC × | Granulocytes × | PLT × | HTC | RBC × |
|---|---|---|---|---|---|---|
| Number | 10/ml | 10/ml | 10/ml | 10/ml | % | 10/ml |
| 1 | 5.6 | 1.4 | 4.2 | 192 | 38.1 | 12.4 |
| 2 | 7.5 | 2.1 | 5.3 | 210 | 37.4 | 3.98 |
| 3 | 4.5 | 1.4 | 3 | 170 | 37.5 | 4.26 |
| 4 | 8 | 1.7 | 6.3 | 240 | 37.6 | 3.95 |
| 5 | 11.3 | 2.9 | 8.5 | 335 | 35.8 | 3.98 |

TABLE 1.2
Hematology data: 7 mPRProtocol

| Sample | WBC × | MC × | Granulocytes × | PLT × | HTC | RBC × |
|---|---|---|---|---|---|---|
| Number | 10/ml | 10/ml | 10/ml | 10/ml | % | 10/ml |
| 1 | 4.0 | 3.2 | 0.8 | 673 | 2.7 | 0.29 |
| 2 | 5 | 4.5 | 0.6 | 755 | 3.0 | 0.33 |
| 3 | 5.9 | 3.4 | 2.5 | 691 | 2.8 | 0.32 |
| 4 | 7.2 | 6.4 | 0.8 | 964 | 2.8 | 0.32 |
| 5 | 7.8 | 7.4 | 0.4 | 1304 | 2.3 | 0.28 |

TABLE 1.3
Hematology data: 14 mPRProtocol

| Sample | WBC × | MC × | Granulocytes × | PLT × | HTC | RBC × |
|---|---|---|---|---|---|---|
| Number | 10/ml | 10/ml | 10/ml | 10/ml | % | 10/ml |
| 1 | 7.1 | 6.6 | 0.5 | 1136 | 0.8 | 0.08 |
| 2 | 12.5 | 11.6 | 0.8 | 1202 | 1.1 | 0.14 |
| 3 | 13.7 | 12.4 | 1.3 | 1072 | 1.9 | 0.21 |
| 4 | 7.7 | 6.9 | 0.8 | 1524 | 0.9 | 0.1 |
| 5 | 15.3 | 14.1 | 1.2 | 1866 | 1.1 | 0.11 |

TABLE 1.4
Platelet Yield (% recovery)

| Sample Number | 14mPRP | 7mPRP |
|---|---|---|
| 1 | 82% | 47 |
| 2 | 86% | 47 |
| 3 | 82% | 55 |
| 4 | 83% | 54 |
| 5 | 67% | 56 |
| Mean | 81% | 49 |
| STDEV | 6% | 8 |

TABLE 1.5
Mononuclear Cell Yield (% recovery)

| Sample Number | 14mPRP | 7mPRP |
|---|---|---|
| 1 | 31% | 65% |
| 2 | 28% | 83% |
| 3 | 33% | 116% |
| 4 | 50% | 53% |
| 5 | 37% | 59% |
| Mean | 33% | 70% |
| STDEV | 10% | 25% |

TABLE 1.5
Mononuclear Cell Yield (% recovery)

| Sample Number | 14mPRP | 7mPRP |
|---|---|---|
| 1 | 31% | 65% |
| 2 | 28% | 83% |
| 3 | 33% | 116% |
| 4 | 50% | 53% |
| 5 | 37% | 59% |
| Mean | 33% | 70% |
| STDEV | 10% | 25% |

TABLE 1.6
Granulocyte Yield (% recovery)

| Sample Number | 14mPRP | 7mPRP |
|---|---|---|
| 1 | 2% | 3% |
| 2 | 2% | 1% |
| 3 | 6% | 11% |
| 4 | 2% | 2% |
| 5 | 2% | 1% |
| Mean | 2% | 3% |
| STDEV | 1% | 4% |

TABLE 1.8
pH

| Sample Number | PRProtocol |
|---|---|
| 1 | 6.8 |
| 2 | 6.8 |
| 3 | 6.7 |
| 4 | 6.9 |
| 5 | 7 |
| Mean | 6.9 |
| STDEV | 0.1 |

### Discussion

Based on the data yielded from the samples (n=5). It shows statistically significant difference <0.01 between the baseline, 14mPRP and 7mPRP.

Classification of PRProtocol:
14mPRP: PAW: P3Bβ
7mPRP: PAW: P2Aβ

Although the embodiments have been described in detail, it should be understood that various changes, substitutions and alterations can be made herein. Moreover, the scope of the present application is not intended to be limited to the particular embodiments or examples described in the specification. As can be understood, the examples described above and illustrated are intended to be exemplary only.

For example, the present invention contemplates that any of the features shown in any of the embodiments described herein, may be incorporated with any of the features shown in any of the other embodiments described herein, and still fall within the scope of the present invention.

1. A kit for preparing a plasma injectate for, the kit comprising:
a. at least one blood collection container, each containing an anticoagulant, for receiving a blood sample;
b. an optional calcium chloride solution, for preparing an activator solution;
c. an activator container for loading the activator solution, the activator container having a first volume; and
d. an injectate syringe having a second volume that is larger than the first volume, the injectate syringe for receiving plasma from the blood sample and for fluid connection with the activator container to transfer at least a portion of the activator solution to the injectate syringe.

2. The kit of claim 1, comprising two blood collection containers.

3. The kit of claim 1, further comprising an adapter for fluid connection of the injectate syringe to the activator container.

4. The kit of claim 1, wherein the calcium chloride solution has a concentration of 50 to 250 mg/ml.

5. The kit of claim 1, wherein the at least one blood collection container comprises an upper chamber of an centrifuge tube and the activator container comprises a lower chamber of the centrifuge tube, and wherein the upper chamber and the lower chamber are separated by a barrier configured to allow mixing of the contents of the upper and lower chambers when centrifuged.

6. The kit of claim 5, wherein the upper or the lower chamber is pre-loaded with the activator solution.

7. The kit of any one of claim 1, further comprising one or more of:
e. at least one extraction needle for coupling to the injectate syringe, for extracting a platelet plasma layer from a centrifuged blood sample
f. at least one venous needle for coupling to the at least one blood collection containers, for drawing blood samples from a patient;
g. at least one venting needles for insertion into the at least one blood collection containers, respectively;
h. saline; and
i. at least one plasma tube for centrifuging the blood sample.

8. The kit of any one of claim 1, wherein the anticoagulant is ACD-A, Sodium Citrate or heparin.

9. The kit of claim 2, wherein the adaptor is a Luer lock adapter.

10. The kit of claim 1, comprising:
i. two blood collection containers, each containing 1 cc per tube ACD-A;
ii. 1 ml of Calcium Chloride at a concentration of 100 mg/ml;
iii. 3 ml activator syringe;
iv. 10 ml injectate syringe;
v. two 3.5 inch 18 gauge needles;
vi. a Luer lock adapter;
vii. the two venous needles; and
viii. two 1.5 inch blunt fill needles.

11. The kit of claim 1, comprising:
i. two blood collection containers, each containing 1 cc per tube ACD-A;
ii. 1 ml of Calcium Chloride at a concentration of 100 mg/ml;
iii. 2 ml activator syringe;
iv. 5 ml injectate syringe;
v. a 3.5 inch 18 gauge needle;
vi. the adapter; and
vii. the two venous needles.

12. The kit of claim 11, wherein i. and iii.-vii. are packaged in a blister pack.

13. The kit of any one of claim 1, wherein the at least one blood collection container each further contain a further calcium chloride solution.

14. A method for preparing a plasma injectate with the kit of claim 1, the plasma injectate for administration to a patient, the method comprising:
loading the activator container with the activator solution;
collecting whole blood sample from the patient in the at least one blood collection container;
centrifuging the whole blood samples;
extracting the platelet plasma layer from the centrifuged blood samples into the injectate syringe;
connecting the injectate syringe having the platelet plasma to the loaded activator syringe; and
transferring at least a portion of the activator solution to the injectate syringe.

15. The method of claim 14, comprising preparing the activator solution by diluting the calcium chloride solution with saline.

16. The method of claim 14, comprising centrifuging the whole blood sample for 7 minutes, wherein the plasma injectate is pro-inflammatory.

17. The method of claim 14, comprising centrifuging the whole blood sample for 14 minutes, wherein the plasma injectate is anti-inflammatory.

18. The method of any one of claim 14, wherein the plasma injectate comprises autologous platelets.

19. The method of any one of claim 14, further comprising injecting the activated platelet plasma into a target area of the patient.
